(19) United States
(12) Patent Application Publication
(10) Pub. No.: US 2025/0177124 A1
(43) Pub. Date: Jun. 5, 2025

(54) PACKAGED PRELOADED INTRAOCULAR LENS SYSTEM (IOL) SYSTEM

(71) Applicant: Bausch + Lomb Ireland Limited, Dublin (IE)

(72) Inventor: Mayank Rameshkumar VYAS, Land O Lakes, FL (US)

(21) Appl. No.: 18/526,210

(22) Filed: Dec. 1, 2023

(51) Int. Cl. A61F 2/16

(52) U.S. Cl. CPC A61F 2/1691

(57) ABSTRACT

A preloaded intraocular lens system (IOL) system, including an inserter element having a IOL operatively disposed relative thereto, a non-vapor-permeable packaging in an open state having the inserter element and the IOL disposed therein, and a vapor permeable packaging having the non-vapor-permeable packaging enclosed therein. The non-vapor permeable packaging is sealed while it is disposed in the vapor permeable packaging, while the vapor permeable packaging is sealed.

### FIELD

Methods and systems for packaging an intraocular lens system in a hydrated state and more particularly, methods and systems for packaging a preloaded inserter element having a IOL operatively disposed relative to the inserter element in a hydrated state.

### BACKGROUND

Medical devices are commonly sterilized using pressure and vapor, most commonly steam. To achieve sterilization of a given device, the sterilization process requires the vapor to reach the device at a selected temperature, pressure, concentration and time. Vapor permeable bags are commonly used to maintain the devices during and after the sterilization process. The bags allow vapor penetration to achieve sterilization and, post sterilization, the bags may be used to keep the device sterile until the bag is opened. Devices that control temperature, pressure, concentration and time are commonly referred to as autoclaves, and flexible autoclave packaging that allow steam penetration and can withstand the temperatures and pressure of the autoclave process are referred to as autoclave bags.

While autoclave bags may be used as packaging in some instances, such bags are not suitable in instances where a sterilized medical device is to be kept hydrated after sterilization because autoclave bags cannot prevent moisture from escaping, thus autoclave bags allow the sterilized device to become dry.

Techniques have been proposed to allow sterilization of medical devices through a steam permeable bag or other container (or a portion of a bag or container) while also providing hydration of the medical devices after the steam sterilization process is completed. According to one technique, a bag includes two sections, a first section that is steam permeable and a second section that is not steam permeable. The medical device is sterilized in the first section and subsequently moved to and sealed in the second section.

According to another technique, a medical device is placed in a bag that is vapor impermeable other than at a vapor permeable head portion formed at the opening of the bag. Prior to sterilization, the medical device is sealed within the non-vapor permeable portion of the bag, with the head portion in fluid communication with the non-gas permeable portion, such a configuration allows the steam to reach the medical device during the sterilization process. After the medical device is sterilized, the bag is sealed at a second location, with the medical device still located within the impermeable portion of the bag. The location of the second seal is selected to prevent fluid communication between the head portion and the vapor impermeable portion.

According to yet another technique, an intraocular lens (IOL) preloaded on an IOL injector is sealed in the interior space of a container that is delimited in part by each of a gas permeable subsection and a gas impermeable subsection which are in fluid communication. The preloaded injector is located within the gas impermeable subsection and is sterilized with gas passing through the gas permeable section. After sterilization, the gas impermeable section is sealed off from the gas permeable section with the preloaded injector remaining inside the gas impermeable section.

While the above techniques allow for sterilization of devices that are to remain hydrated post-sterilization, there remains a need for alternative packaging systems for devices that are to remain hydrated.

### SUMMARY

According to aspects of the present invention, a versatile post-sterilization construction for use with ophthalmic medical devices is obtained. In a first configuration, the construction includes a non-vapor permeable packaging contained within a sealed vapor permeable packaging (e.g., an autoclave bag), a sterilized ophthalmic medical device present within the non-vapor permeable packaging. In a first state, the non-vapor permeable packaging is unsealed thus allowing the vapor to enter the non-vapor permeable packaging to allow sterilization. In a second state, after the non-vapor permeable packaging is sealed (post sterilization), the non-vapor permeable packaging maintains the device in a hydrated state and/or environment.

After the second state is achieved, the sterilized device contained in the packaging may be introduced into a surgical field by opening the outer, vapor permeable packaging within the surgical field. Alternatively, the outer packaging can be opened prior to reaching the surgical field (e.g., at the manufacturing site where sterilization occurs).

In some instances, sealing of the inner non-vapor permeable packaging occurs after sterilization and within the outer vapor permeable packaging, with the inner packaging remaining unattached to the outer packaging. Accordingly, upon opening of the outer packaging, the inner packaging with the ophthalmic medical device contained therein is introduced into the surgical field without the inner packaging having been modified (e.g., cut or broken or torn) thereby facilitating separation of the inner packaging from the outer packaging. In some instances, the device is an inserter (or a portion thereof) having an IOL operatively disposed relative thereto. Alternatively, the device may be a contact lens or another ophthalmic medical device such as a single-use surgical instrument.

Other aspects of the present invention are directed to a method of packaging an ophthalmic medical device. The methods efficiently and effectively provide a structure for maintaining the device in a hydrated state and/or in a moist environment. Methods result in a non-vapor permeable packaging that is disposed in a vapor permeable packaging, the non-vapor permeable packaging having the device disposed therein.

According to an aspect of the invention a preloaded intraocular lens (IOL) system comprises an inserter element having an IOL operatively disposed relative thereto, a non-vapor-permeable packaging in an open state having the inserter element and the IOL disposed therein, and a vapor permeable packaging having the non-vapor-permeable packaging enclosed therein.

In some embodiments, the vapor permeable packaging is sealed closed. The vapor permeable packaging may comprise a window through which an open end of the non-vapor permeable is visible.

In some embodiments, the inserter element is a complete inserter. In some embodiments, the inserter element is an IOL cartridge.

The non-vapor-permeable packaging container may contain water. In some embodiments, the non-vapor-permeable packaging contains 5-10 mL of water.

In some embodiments, the IOL is disposed in a substantially unstressed state. The IOL may be a hydrophobic IOL.

Another aspect of the invention is directed to a packaged preloaded, ophthalmic device system comprising a non-vapor-permeable packaging in an open state having the ophthalmic device disposed therein, and a vapor permeable packaging having the non-vapor-permeable packaging disposed therein.

In some embodiments, the vapor permeable packaging comprises a window through which an open end of the non-vapor permeable is visible. The non-vapor-permeable packaging may contain water.

Yet another aspect of the invention is directed to a method of packaging a preloaded intraocular lens (IOL), comprising a step of sealing a non-vapor permeable packaging that is disposed in a sealed vapor permeable packaging, the non-vapor permeable packaging having an inserter element disposed therein with an IOL operatively disposed relative to the inserter element.

In some instances, prior to the step of sealing, the inserter element and the IOL are sterilized by applying steam to the vapor permeable packaging. The sterilization may occur within an autoclave.

In some instances, liquid water is added into the non-vapor permeable packaging prior to the sealing step (e.g., 5-10 mL). The step of sealing may be performed while viewing an end of the non-vapor permeable packaging through a window of the vapor permeable packaging.

The inserter element may be a complete inserter. The IOL may be in a substantially unstressed state. The IOL may be a hydrophobic IOL.

These and other aspects of the present invention will become apparent upon a review of the following detailed description and the claims appended thereto.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1 is a schematic illustration of an example of a packaged preloaded intraocular lens system (IOL) system comprising an inserter element having an IOL 120 operatively disposed relative thereto, a non-vapor permeable packaging in an open state containing the inserter element and IOL, and a vapor permeable packaging in a closed state containing the non-vapor permeable packaging;

FIG. 2 is a schematic illustration of an example of a packaged preloaded intraocular lens system (IOL) system comprising an inserter element 110 having an IOL 120 operatively disposed relative thereto, a non-vapor permeable packaging in a sealed state containing the non-vapor permeable packaging, and a vapor permeable packaging containing the non-vapor permeable packaging; and

FIG. 3 is a flowchart illustrating an example of method of packaging a preloaded intraocular lens (IOL) inserter according to the present invention.

### DETAILED DESCRIPTION

Aspects of the invention will be further described with reference to the following specific examples. It is understood that these examples are given by way of illustration and are not meant to limit the claims to any specific example unless language of the claims explicitly does so. For instance, aspects of the invention will be described below with reference to preloaded IOL system; however, aspects of the invention may include other ophthalmic devices, such as a contact lens or a single use ophthalmic surgical instrument.

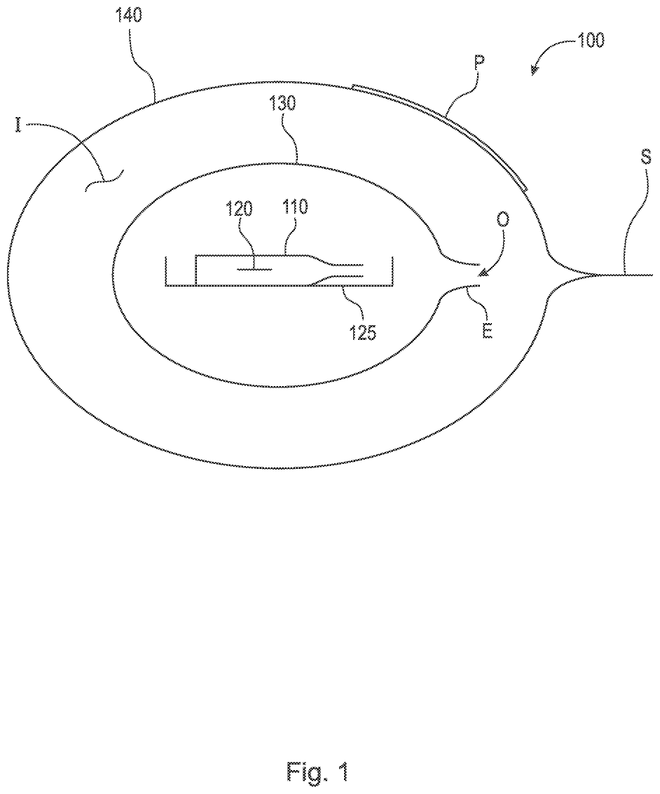

FIG. 1 is a schematic illustration of an example of a packaged preloaded intraocular lens system (IOL) system 100 comprising an inserter element 110 having an IOL 120 operatively disposed relative thereto, a non-vapor permeable packaging 130 in an open state, and a sealed vapor permeable packaging 140.

Inserter element 110 may be a complete inserter or a portion of the inserter (e.g., an IOL cartridge) that can be attached to a remaining portion to form a compete inserter. An operatively disposed IOL (also referred to herein simply as a lens) is positioned relative to the inserter element such that actuation of the inserter element and/or actuation the remaining portion results in the lens being ready for insertion into an eye. For example, actuation may include rotation of the inserter element or a portion of the remaining portion to fold the lens, lateral compression to fold the lens, actuation of a plunger that is oriented along a longitudinal axis of the complete inserter such that, as the lens is moved into the eye, features along the walls of the lumen of the complete inserter fold the lens. It is typically advantageous that an operatively disposed lens is in a substantially unstressed lens state when packaged (e.g., where the only stress is caused by deformation due to gravity). The lens may be positioned relative to the inserter element in any suitable manner, for example, with the lens resting on a surface of the inserter element or by supporting haptics of the lens or edges of the optic of the lens.

As shown in FIG. 1, vapor permeable packaging 140 has the non-vapor-permeable packaging 130 enclosed therein. Vapor permeable packaging 140 is sealed after non-vapor packaging 130 is located therein to form an enclosed interior I. Typically, to form enclosed interior I, the sealing occurs at a single end S (i.e., the vapor permeable packaging 140 is a bag). Non-vapor permeable packaging 130 is open while the vapor is applied such that the vapor permeating the vapor permeable packaging 140 enters the non-vapor permeable packaging 130 through the open end O. Vapor permeable packaging 140 is constructed to allow a sterilizing vapor to penetrate therethrough and enter interior I sufficiently to sterilize inserter element 110 and IOL 120.

Sterilization using steam that is generated within an autoclave is well known. Sterilization in an autoclave is accomplished by controlling vapor concentration (e.g., humidity), temperature, pressure, and time. In such instances, vapor permeable packaging 140 may be an autoclave bag. Autoclave bags are commonly made of high-density polyethylene (HDPE), Polypropylene (PP), Polyamide, paper, or a combination thereof. For example, a vapor permeable material may be a film of suitable thickness or a non-woven fabric (such as high-density polyethylene available from Dupont™ under the name Tyvek®).

The humidity of the vapor during the steam sterilization is typically selected such that lens 120 is sufficiently hydrated upon return of the vapor to room temperature after the sterilization process is completed, and the non-vapor permeable packaging is sealed in the manner set forth below. It will be appreciated that condensation of the steam after the vapor reaches room temperature will result in a substantial amount of liquid water, including any water absorbed by the lens (e.g., a few droplets of water or about 5-10 ml of water); however, additional liquid water can be added within the non-vapor permeable packaging prior to sealing vapor permeable packaging 140 as deemed suitable. The additional water may be determined in part by a desire to increase the shelf life of the packaged lens.

Although steam sterilization was discussed above, other possible sterilizing agents include ethylene oxide, ozone, hydrogen peroxide or combinations of the sterilizing agent(s) with steam. Similar to sterilization using steam, vapor permeable packaging 140 when using a sterilizing agent other than steam is selected which is suitably permeable to the selected sterilizing agent. The entirety of the vapor permeable packaging may be permeable to the sterilizing agent or only a portion of the vapor permeable packaging provided sufficient vapor reaches the lens and/or inserter to achieve sterilization.

As discussed in greater detail below, it is typically advantageous that non-vapor permeable packaging 140 includes at least a portion P that is transparent such that open end O of the non-vapor permeable packaging 130 is visible (when non-vapor permeable packaging 130 is disposed within vapor permeable packaging 140) to facilitate sealing of non-vapor permeable packaging 130.

Non-vapor-permeable packaging 130 has inserter element 110 and the IOL 120 disposed therein. Non-vapor permeable packaging 130 is sealed with moisture from the sterilization process still in the packaging, such that IOL 120 is kept hydrated after sealing. The goal is to keep IOL 120 hydrated until the packaging is opened to prepare IOL 120 for implantation; however, it is understood that non-vapor permeable packaging allows escape of some moisture resulting in a finite shelf life of a packaged lens. A typical shelf life is 3-5 years however a packaging may be designed to provide any suitable shelf life.

The non-vapor permeable packaging operates to substantially prevent water vapor and liquid water from traversing the non-vapor permeable packaging and operates as a sterility barrier thereby keeping the device packaged therein sterile. For example, the non-vapor permeable packaging may be made of a polyester film, stretched polyethylene terephthalate (PET) (e.g., Mylar® available form Dupont™), a metal foil (e.g., aluminum foil), or a plastic such as polypropylene coated with a metal (e.g., aluminum). The term non-vapor permeable packaging refers to materials having a water vapor permeability of less than 0.5 g/m²/24 h, and in some instances less than 0.1 g/m²/24 h.

IOL 120 may be any suitable conventional hydrophobic or hydrophilic IOL. Inserter element 110 may be supported by a tray 125 such as blister packing.

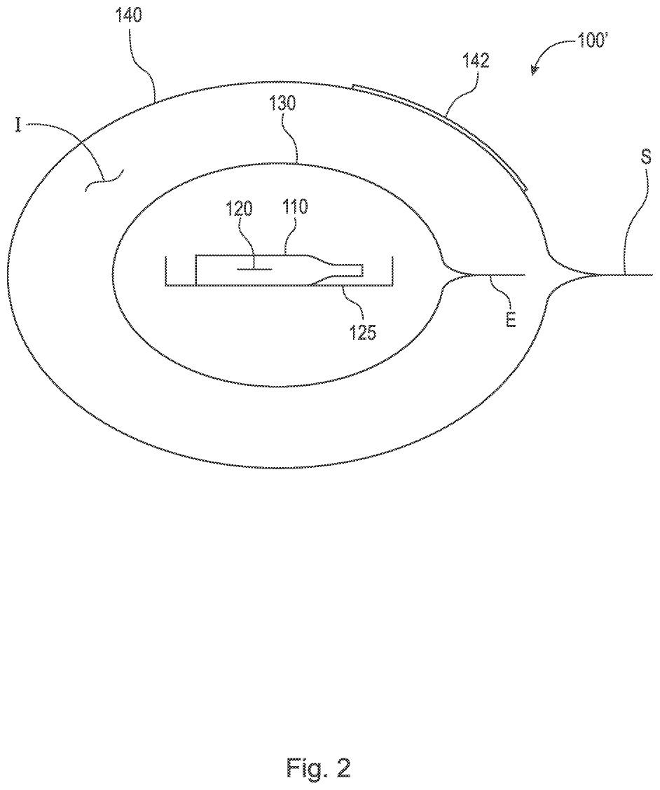

FIG. 2 is a schematic illustration of an example of a packaged preloaded intraocular lens system (IOL) system 100′ shown in FIG. 1 with non-vapor permeable packaging 130 in a closed (i.e., sealed) state.

After sterilization of inserter element 110 and IOL 120 and while the humidity of the sterilization process is within interior I, non-vapor permeable packaging 130 is sealed closed (as shown in FIG. 2) trapping water molecules of the steam therein to maintain the IOL in a hydrated state.

As the inventors unexpectedly discovered, it is possible to seal the non-vapor permeable packaging 130 while the non-vapor permeable packaging 130 is located within a sealed vapor permeable packaging 140 without adhering the non-vapor permeable packaging 130 to vapor permeable packaging 140. To seal the non-vapor permeable packaging 130 while the non-vapor permeable packaging 130 is located in the vapor permeable packaging 140, heat (e.g., from a bag sealer) is applied at an end E at a temperature that welds the non-vapor permeable packaging 130 while not welding the vapor permeable packaging 140. Typically, avoiding adherence of packaging 130 to packaging 140 facilitates removal of the packaging 140 from packaging 130; however, in some instances a higher temperature may be applied such that packaging 140 and packaging 130 are welded together at sealing.

For example, vapor permeable packaging 140 may be a pouch made of Tyvek on one side and polyethene (which is clear to form a window extending the length of the pouch to facilitate sealing of the non-vapor permeable bag) on other side as is conventionally known; the non-vapor permeable bag 130 may be polypropylene coated on an outer surface with an aluminum foil. For such a configuration, the temperature of the bag sealer may be in a range of 375 F to 425 F to melt the polypropylene of the inner, non-vapor permeable bag without adhering the non-vapor permeable bag to the vapor permeable bag.

Typically, non-vapor permeable packaging 130 is bag-shaped and the bag sealer (having a linear heating element) is applied across end E to seal opening O closed. As discussed above, vapor-permeable packaging 140 may be provided with a transparent portion 142 to allow viewing of non-vapor permeable packaging 130 and alignment of the heating element with end E of non-vapor permeable bag 130.

Vapor-permeable outer packaging 140 (such as a conventional autoclave bag) is permeable to steam and impermeable to biologic materials, in some instances making it advantageous to employ vapor permeable packaging 140 as secondary package, with non-vapor permeable packaging 130 forming a primary package. In other instances, it may be desirable to open vapor permeable packaging 140 and remove non-vapor permeable packaging 130 with the IOL and inserter element inside, prior to sale; optionally, after the non-vapor permeable packaging 130 is removed from the vapor permeable bag 140, the non-vapor permeable bag 130 may be packaged in another secondary package (not shown).

Aspects of the invention are directed to a method of packaging a preloaded intraocular lens (IOL) inserter, comprising sealing a non-vapor permeable packaging that is disposed in a vapor permeable packaging, the non-vapor permeable packaging having the preloaded IOL inserter disposed therein with an IOL operatively disposed relative inserter.

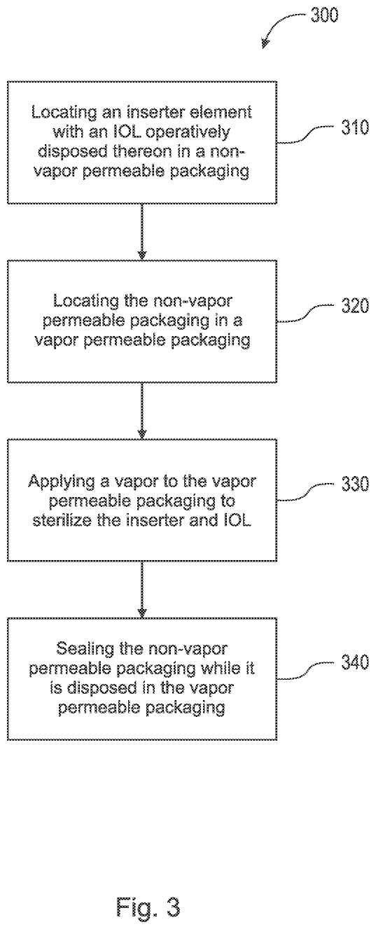

FIG. 3 is a flowchart 300 illustrating an example of method of packaging a preloaded intraocular lens (IOL) inserter according to the present invention. At step 310, an inserter element having an IOL operatively disposed therein is located in a non-vapor permeable packaging (typically a bag).

At step 320, non-vapor permeable packaging (including inserter element having the IOL operatively disposed relative to the inserter element) is located in a vapor permeable packaging (typically a bag) with the non-vapor permeable packaging being in an open state.

At step 330, the vapor is applied to the vapor permeable packaging to sterilize the inserter and the IOL. The vapor passes through (a wall of) the vapor permeable packaging and enters the non-vapor permeable packaging to sterilize the IOL and the inserter element. The vapor may be steam, another sterilizing agent, or a combination of steam with another sterilizing agent.

At step 340, the non-permeable packaging is sealed while it is disposed in the vapor permeable packaging, the inserter and IOL disposed in the non-permeable packaging. The sealing process is typically accomplished with the non-permeable packaging not being adhered to the vapor permeable packaging as a result of the sealing. If the vapor is steam, sealing occurs while water molecules of the steam are present within the interior of the non-vapor permeable packaging such that water molecules of the steam are trapped with the non-vapor permeable packaging to maintain the IOL in a hydrated state while the inserter element and IOL are packaged.

The non-vapor permeable packaging can be extracted from the vapor permeable packaging during manufacture, or the vapor permeable packaging can be used as a secondary packing (maintaining the exterior of the non-vapor permeable packaging sterile during transport to a surgical site). Regardless of when the vapor permeable packaging is removed, it will be appreciated that the non-vapor permeable packaging can be unmodified by the removal because the non-permeable packaging is not adhered to the vapor permeable packaging.

Although the apparatus and methods of the invention set forth above are described with reference to an IOL and injector element, the device could be a contact lens or an ophthalmic surgical instrument, typically an instrument that is for single use.

Although various embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A preloaded intraocular lens (IOL) system, comprising:
an inserter element having an IOL operatively disposed relative thereto;
a non-vapor-permeable packaging in an open state having the inserter element and the IOL disposed therein; and
a vapor permeable packaging having the non-vapor-permeable packaging enclosed therein.

2. The system of claim 1, wherein the vapor permeable packaging is sealed closed.

3. The system of claim 1, wherein the vapor permeable packaging comprises a window through which an open end of the non-vapor permeable is visible.

4. The system of claim 1, wherein the inserter element is a complete inserter.

5. The system of claim 1, wherein the inserter element is an IOL cartridge.

6. The system of claim 1, wherein the non-vapor-permeable packaging container contains 5-10 ml of water.

7. The system of claim 2, wherein the non-vapor-permeable packaging contains 5-10 mL of water.

8. The system of claim 1, wherein the IOL is disposed in a substantially unstressed state.

9. The system of claim 1, wherein the IOL is a hydrophobic IOL.

10. A packaged preloaded ophthalmic device system, comprising:
a non-vapor-permeable packaging in an open state having the ophthalmic device disposed therein; and
a vapor permeable packaging having the non-vapor-permeable packaging disposed therein.

11. The system of claim 10, wherein the vapor permeable packaging comprises a window through which an open end of the non-vapor permeable is visible.

12. The system of claim 10, wherein the non-vapor-permeable packaging contains water.

13. A method of packaging a preloaded intraocular lens (IOL), comprising:
sealing a non-vapor permeable packaging that is disposed in a sealed vapor permeable packaging, the non-vapor permeable packaging having an inserter element disposed therein with an IOL operatively disposed relative to the inserter element.

14. The method of claim 13 wherein, prior to the step of sealing, the inserter element and the IOL are sterilized by applying steam to the vapor permeable packaging.

15. The method of claim 14, wherein the sterilization occurs within an autoclave.

16. The method of claim 15, wherein liquid water is added into the non-vapor permeable packaging prior to the sealing step.

17. The method of claim 13, wherein the step of sealing is performed while viewing an end of the non-vapor permeable packaging through a window of the vapor permeable packaging.

18. The method of claim 13, wherein the inserter element is a complete inserter.

19. The method of claim 13, wherein the IOL is in a substantially unstressed state.

20. The method of claim 13, wherein the IOL is a hydrophobic IOL.
